(19) United States
(12) Patent Application Publication
(10) Pub. No.: US 2025/0143722 A1
(43) Pub. Date: May 8, 2025

(54) TOOL FOR CUTTING BONE FOR SURGICAL USE

(71) Applicant: RAZOR Medical Instruments, Inc., Warsaw, IN (US)

(72) Inventors: George B. Cipolletti, Duxbury, MA (US); Nader Amir Nassif, Costa Mesa, CA (US); Eeuwout A.C. Hoogendijk, Duxbury, MA (US)

(21) Appl. No.: 18/939,330

(22) Filed: Nov. 6, 2024

Related U.S. Application Data
(60) Provisional application No. 63/547,519, filed on Nov. 6, 2023.

(51) Int. Cl. A61B 17/16

(52) U.S. Cl. CPC A61B 17/1617

(57) ABSTRACT

The disclosure is directed to a reamer device for surgical procedures. The reamer device comprises a centralizer with a circular body and an exterior surface. The centralizer defines an interior cavity, wherein the centralizer comprises an apex region located at a distal region of the centralizer. The apex region defines an alignment protrusion comprising at least one alignment rib oriented adjacent to an alignment slot. The device further comprises a plurality of cutting ribs. Each cutting rib comprises an arcuate portion and protrudes from an exterior surface of the centralizer. The centralizer defines a slot adjacent to each cutting rib of the plurality of cutting ribs. The slot defines a conduit from the exterior surface of the centralizer to the interior cavity of the centralizer. The arcuate portion of the cutting rib is formed into a profile that follows the exterior surface of the centralizer.

### CROSS RELATED APPLICATIONS

The present disclosure is related and claims priority under 35 U.S.C. 119(e) to U.S. Provisional Patent Application No. 63/547,519 filed on Nov. 6, 2023, the disclosure of which is hereby incorporated by reference in its entirety for all purposes.

### TECHNICAL FIELD

The present disclosure relates to cutting tools used for cutting bone during joint replacement. One particular application for the cutting tool is to be used for cutting the cavity of the acetabulum when replacing the hip joint by a prosthetic cup.

### BACKGROUND

Tools that are used in surgical procedures for cutting bone may yield to produce an appropriate dimension for an acetabular cavity. Excluding preparation time for the usage of the tool, the requirement for the reamer to produce a hemispherical cavity can be difficult to produce. Additionally to extended preparation times, the size of an acetabulum reamed with a blunt reamer may not accurately represent the desired size. This inaccuracy may then adversely affect implant selection or implantation and complicate post-surgery recovery, reducing the overall effectiveness of the procedure. For example, reamer slippage can cause the reamer to divert from the axis preventing a uniform hemispherical cavity from being generated in the cutting surface.

### SUMMARY

In some embodiments, a device includes a cutting rib, the cutting rib including an arcuate portion having an outer sharp edge and a centralizer configured to receive the cutting rib through a radial slot, the radial slot forming a gap with the cutting rib. The arcuate portion of the cutting rib is formed into a profile that follows a surface of the centralizer, the outer sharp edge of the arcuate portion of the cutting rib has a wavy profile that forms a tunnel in the gap between the centralizer and the cutting rib, and the outer sharp edge of the arcuate portion of the cutting rib protrudes outwardly from the surface of the centralizer.

### BRIEF DESCRIPTION OF THE DRAWINGS

FIG. 1A is a perspective view of a cutting rib assembly, according to some embodiments.

FIG. 1B illustrates a top view of the cutting rib assembly, according to some embodiments.

FIG. 2A illustrates a partial view of a distal region of the centralizer assembly.

FIG. 2B illustrates a magnified view of the partial view in FIG. 2A.

In the figures, elements having the same or similar reference numeral have the same or similar functionality or configuration, unless expressly stated otherwise.

### DETAILED DESCRIPTION

Embodiments disclosed herein include a tool for cutting bone that can maintain alignment when creating the cavity to ensure that the cavity is uniformly dimensioned. In some embodiments, the apex of the cutting tool can comprise a different cutting profile to aid the cutting tool in maintaining alignment along the rotation axis for the duration of the reaming process. As a result, the tool can provide an accurate and predictable cutout shape (e.g., the acetabular socket in the pelvic area, or hip), thus ensuring predictable and accurate cutting. Further, tools and reamers as disclosed herein can endure high instantaneous torque during reaming and shaping of bone and other substrates.

During hip replacement surgery, the acetabulum socket in the pelvic bone is prepared for receiving an acetabular cup. Accordingly, the medial floor of the acetabulum socket is identified so that a first acetabular reamer as disclosed herein (of approximately 6 mm or so smaller than a final diameter acetabular reamer) is used to ream medially to the acetabular floor. The initial reamed cavity acts as a centralizer for successive acetabular reamers consistent with the present disclosure. The successive acetabular reamers, of increased dimension, expand the diameter of the acetabular socket. Sequential reamers, as disclosed herein, may be provided in 1 or 2 mm increments in diameter, and are used to further expand the peripheral rim of the socket, while preserving the integrity of the medial and lateral walls. With each instance of expanding the cavity, the acetabular reamer must maintain the cutting axis of the previous instance to maintain a uniform acetabular cup (cavity). Embodiments as disclosed herein avoid the above problems by defining a region at the apex of the reamer with a different cutting profile to reduce slippage. The reamer profile at the apex defines an angle with respect to the cutting axis to aid in anchoring the reamer during the cutting process.

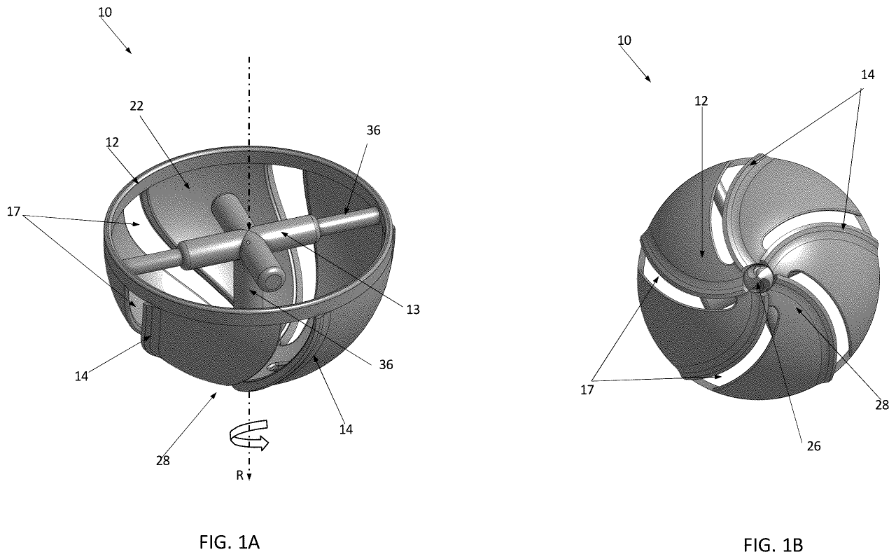

FIG. 1A is a perspective view of a cutting rib assembly 10, according to some embodiments. Cutting rib assembly 10 may include a centralizer 12 and cutting ribs 14. In some embodiments, cutting rib assembly 10 may be selectively disposable. In some embodiments, centralizer 12 may have a hemispherical shape to cut into a substrate shaped at least partially in a spherical portion (e.g., for hip replacement applications). Other geometries may be better suited in different applications. For example, in some embodiments, centralizer 12 may have a radial shape adjusted to more accurately guide cutting ribs 14 in selected areas of the bone socket. Other shapes may be suitable for different applications, such as a more planar shape for “flattening” bone applications, or other shapes more appropriate such as in ankle surgery, and the like. In some embodiments, it is desirable for centralizer 12 and cutting rib assembly 10 to have an axisymmetric shape, where the symmetry axis is the rotation axis of the assembly. More generally, the centralizer 12 has a shape configured to form a cutout portion consistent with the acetabular region of a patient's hip or any other anatomic area.

Cutting rib assembly 10 may include multiple cutting ribs 14. In some embodiments, multiple cutting ribs 14 are assembled symmetrically about an axis of rotation (R) of cutting rib assembly 10, wherein centralizer 12 includes a radial slot 17 for each of cutting ribs 14. The cutting ribs 14 can further comprise chip breakers on the surface of the cutting ribs 14 to create smaller bone particles.

In some embodiments, cutting ribs 14 may have a wavy shape, or a shape with alternating forward cutting edges. The wavy shape of cutting ribs 14 further stiffens ribs and provides a guide shaved bone into centralizer 12 for collecting shaved bone. Accordingly, a wavy profile of the blade portion in cutting rib 14 provides strength to an otherwise potentially weak structure, wherein the blade of cutting rib 14 may be as thin as about 1 mm, or even less. In some instances, the wavy shape can include a shape having angulated edges and alternating planar cutting surfaces, and in some instances, the wavy shape can include curvatures. For example, in some embodiments, a wavy edge of cutting rib 14 may have an “S” shape, or close to an “S” shape with soft turning of the cutting blade. Further, in some embodiments, the wavy edge of cutting rib 14 may have a square profile, with sharp turning edges of the cutting blade. Moreover, in some embodiments, the wavy edge of cutting rib 14 may slightly change profile along the distal direction of cutting rib 14. For example, the wavy edge may have a softer, “S”-type configuration in the proximal area, and a more square type configuration in the distal area (e.g., close to the apex of centralizer 12).

FIGS. 1A-1B illustrate a perspective view of the centralizer and a top view of the centralizer. The proximal part of the centralizer can include a crossbar 13 so that cutting assembly 10 may be driven externally by a rotary machine (not shown). For example, the crossbar is configured to couple with a reamer handle configured to provide a rotating motion about a symmetry axis of the centralizer 12. Crossbar 13 is configured to withstand a high torque from a reamer handle driving a rotation of the cutting rib assembly against the substrate resistance. Embodiments as disclosed herein provide sharp cutting edges in wavy radial slots 17 that reduce the substrate resistance, thereby relaxing the stress constraint for crossbar 13. Accordingly, crossbars 13 are resistant to breakage from cutting rib assembly 10. For example, directly coupling reamer handle (not shown) with crossbar 13 minimizes functional assembly tolerances, as the relative position of centralizer 12 becomes less critical to performance. The crossbar 13 can be coupled to a side interior surface of the centralizer 12 and to an interior surface of the centralizer proximate to the apex region of the centralizer via connecting rods 36.

In some embodiments, centralizer 12 includes a partially hemispherical hollow body of appropriate thickness usually less than 5 mm thick. The hollow body includes a proximal opening and a distal opening with three or more radial slots 17 laterally placed. The cutting rib assembly is made of four (4) or more cutting ribs 14; the edge of each cutting rib 14 is sharpened on the sides that would be cutting bone. Radial slots 17 fit cutting ribs 14 that push on one side of centralizer 12 as they rotate. Radial slots 17 provide a passageway for bone chips into the interior cavity 22 of centralizer 12. The bone chips are collected inside and can later be used to augment bone deficiencies. In a further aspect, the cutting ribs can comprise a dam oriented at an edge surface of the cutting ribs to aid in guiding the bone chips into the interior cavity.

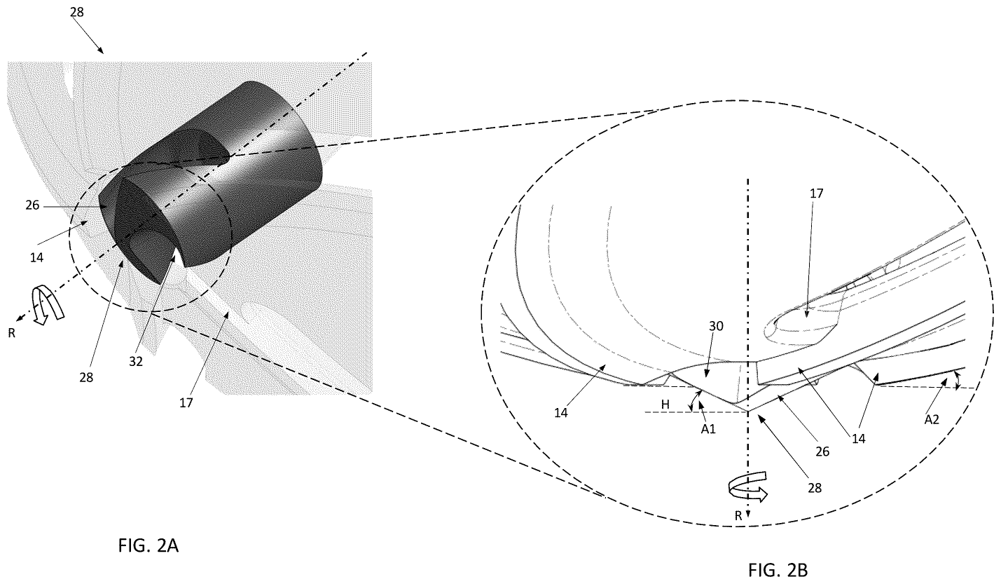

FIG. 2A illustrates a partial view of distal region (apex) 28 of the cutting rib assembly 10. FIG. 2B illustrates a magnification of the partial view. The alignment protrusion 26 is located at the distal end 24 of the centralizer. In a further aspect, the alignment protrusion 26 can be oriented at the apex 28 of the centralizer at the distal end. The alignment protrusion 26 can comprise an alignment rib 30, wherein the alignment rib can be configured to generate a notch (not shown) in the cutting surface (bone) during the reaming process. The notch generated by the alignment rib 30 can be used to reduce the surface area between the centralizer and cutting surface, anchoring the centralizer and mitigate slippage as the centralizer 12 rotates around a rotation axis (R). In a further aspect, the centralizer 12 can be symmetric around the rotation axis. The alignment protrusion 26 can extend further from the surface of the centralizer 12 than the cutting ribs 14 such that there is a height (H) between the tip of the alignment protrusion 26 and maximum height of a cutting rib 14. The alignment protrusion 26 can comprise at least one alignment rib 30 oriented at an angle (A1) that is different from the angle (A2) such that the respective angles have different measurements. Adjacent to the alignment protrusion 26 is an alignment slot 32. The alignment 32 slot can be a conduit that allows extracted cutting surface material to pass from the exterior surface of the centralizer to the interior cavity 22 of the centralizer, similar to the functionality of the radial slots 17.

To ease cutting the bone, surgeons use powered reamer drivers. They can spin as fast as 750 revolutions per minute (RPM). In some embodiments, a preferred rotational speed of reamers and tools as disclosed herein may be 350 RPM, or even lower than 350 RPM. More generally, surgeons may use reamers and tools as disclosed herein at speeds between about 350 RPM and about 750 RPM. At such speeds, chips have to be effectively removed from the cutting surface of the rib and the cavity, otherwise they will be clogging the cutting edge and hinder cutting and advancement of the reamer. If the slot in the centralizer is in close proximity or attached to the cutting rib, it would then hinder the passage of bone chips into the centralizer. Slots have to be distanced radially from the cutting edge sufficiently to allow the passage of soft tissue, cartilage and bone chips.

When reaming bone, friction between the cutter edge and bone generates heat. It is generally accepted that the critical level at which thermal necrosis appears in bone is when the bone is exposed to a temperature of around 56° C., over a time span exceeding 10 seconds. At this temperature, the denaturation point of alkaline phosphatase is reached, which means that the change in properties is directly attributed to the reorientation of collagen molecules and hydroxyapatite. Healing, following bone surgery, may be delayed or even prevented if the bone cells are injured by thermal necrosis resulting from frictional heat generated during surgical preparation.

While the above description contains many specifics, these should not be construed as limitations on the scope of the disclosure, but rather as exemplifications of one or another preferred embodiment thereof. In some instances, some features of the present invention may be employed without a corresponding use of the other features. Accordingly, it is appropriate that the foregoing description be construed broadly and understood as being given by way of illustration and example only, the spirit and scope of the invention being limited only by the appended claims when filed.

Multiple variations and modifications are possible in the embodiments of the invention described here. Although certain illustrative embodiments of the invention have been shown and described here, a wide range of modifications, changes, and substitutions is contemplated in the foregoing disclosure. While the above description contains many specifics, these should not be construed as limitations on the scope of the invention, but rather as exemplifications of one or another preferred embodiment thereof. In some instances, some features of the present invention may be employed without a corresponding use of the other features. Accordingly, it is appropriate that the foregoing description be construed broadly and understood as being given by way of illustration and example only, the spirit and scope of the invention being limited only by the appended claims.

The foregoing description is provided to enable a person skilled in the art to practice the various configurations described herein. While the subject technology has been particularly described with reference to the various figures and configurations, it should be understood that these are for illustration purposes only and should not be taken as limiting the scope of the subject technology.

There may be many other ways to implement the subject technology. Various functions and elements described herein may be partitioned differently from those shown without departing from the scope of the subject technology. Various modifications to these configurations will be readily apparent to those skilled in the art, and generic principles defined herein may be applied to other configurations. Thus, many changes and modifications may be made to the subject technology, by one having ordinary skill in the art, without departing from the scope of the subject technology.

As used herein, the phrase “at least one of” preceding a series of items, with the term “and” or “or” to separate any of the items, modifies the list as a whole, rather than each member of the list (e.g., each item). The phrase “at least one of” does not require selection of at least one of each item listed; rather, the phrase allows a meaning that includes at least one of any one of the items, and/or at least one of any combination of the items, and/or at least one of each of the items. By way of example, the phrases “at least one of X, Y, and Z” or “at least one of X, Y, or Z” each refer to only X, only Y, or only Z; any combination of X, Y, and Z; and/or at least one of each of X, Y, and Z.

Furthermore, to the extent that the term “include,” “have,” or the like is used in the description or the claims, such term is intended to be inclusive in a manner similar to the term “comprise” as “comprise” is interpreted when employed as a transitional word in a claim. The word “exemplary” is used herein to mean “serving as an example, instance, or illustration.” Any embodiment described herein as “exemplary” is not necessarily to be construed as preferred or advantageous over other embodiments.

A reference to an element in the singular is not intended to mean “one and only one” unless specifically stated, but rather “one or more.” The term “some” refers to one or more. All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description.

While certain aspects and embodiments of the subject technology have been described, these have been presented by way of example only, and are not intended to limit the scope of the subject technology. Indeed, the novel methods and systems described herein may be embodied in a variety of other forms without departing from the spirit thereof. The accompanying claims and their equivalents are intended to cover such forms or modifications as would fall within the scope and spirit of the subject technology.

What is claimed is:

1. A device, comprising:
a centralizer comprising a circular body comprising an exterior surface and defines an interior cavity, wherein the centralizer comprises an apex region located at a distal region of the centralizer such that the apex region defines an alignment protrusion comprising at least one alignment rib oriented adjacent to an alignment slot; and
a plurality of cutting ribs wherein each cutting rib comprises an arcuate portion and protrudes from an exterior surface of the centralizer, wherein:
the centralizer defines a slot adjacent to each cutting rib of the plurality of cutting ribs, wherein the slot defines a conduit from the exterior surface of the centralizer to the interior cavity of the centralizer, and
the arcuate portion of the cutting rib is formed into a profile that follows the exterior surface of the centralizer.

2. The device of claim 1, wherein the alignment protrusion and the centralizer are concentric with respect to an axis of rotation.

3. The device of claim 1, wherein the plurality of cutting ribs are oriented at a first angle relative to the exterior surface of the centralizer and the at least one alignment rib is oriented at a second angle relative to the exterior surface of the centralizer.

4. The device of claim 1, wherein each of the plurality of cutting ribs is arcuate along the exterior surface of the centralizer.

5. The device of claim 1, wherein a distal end of the plurality of cutting ribs terminates at an offset distance from the alignment protrusion.

6. The device of claim 5, wherein the alignment protrusion extends further from the exterior surface than the plurality of cutting ribs at the distal end.

7. The device of claim 1, wherein the centralizer has a hemi-spherical shape to cut into a substrate shaped at least partially in a spherical portion.

8. The device of claim 1, wherein the centralizer has a shape configured to form a cutout portion consistent with the acetabular region of a patient's hip.

9. The device of claim 1, further comprising a crossbar at a proximal end of the centralizer, wherein the crossbar is configured to couple with a reamer handle configured to provide a rotating motion about a symmetry axis of the centralizer.

10. The device of claim 1, further comprising a geometry at a proximal end of the cutting rib, wherein the geometry is configured to couple with a reamer handle configured to provide a rotating motion about a symmetry axis of the centralizer.
